SEQUENCE LISTING (47 sequences)

SEQ ID NO: 1 - DNA, 40 bp, Artificial Sequence
  Synthetic (HFL aptamer)
ataggacggatgagcgtccggccggatgggttttccatca

SEQ ID NO: 2 - DNA, 40 bp, Artificial Sequence
  Synthetic (HFL aptamer)
ctgggacgcggatgagtccgctccaccagccatccatgta

SEQ ID NO: 3 - DNA, 40 bp, Artificial Sequence
  Synthetic (HFL aptamer)
ttaggacgcgaagatgtccttgcaagggctcatcgcatca

SEQ ID NO: 4 - DNA, 40 bp, Artificial Sequence
  Synthetic (HFL aptamer)
atgggacgaccctgtgtccctatatgcgcggagagccatc

SEQ ID NO: 5 - DNA, 40 bp, Artificial Sequence
  Synthetic (HFL aptamer)
cttggacgccctcgtgtcccttgctccttcacatacgcaa

SEQ ID NO: 6 - DNA, 40 bp, Artificial Sequence
  Synthetic (HFL aptamer)
gctggacgttcgtgggtcccattctgaagcctaagactgg

SEQ ID NO: 7 - DNA, 40 bp, Artificial Sequence
  Synthetic (HFL aptamer)
gagggacgaacaagcgtccagcggttatgcaccttcggta

SEQ ID NO: 8 - DNA, 40 bp, Artificial Sequence
  Synthetic (HFL aptamer)
tacggacgcacctccgtccggttctcagcgtggggcgcac

SEQ ID NO: 9 - DNA, 40 bp, Artificial Sequence
  Synthetic (HFL aptamer)
gcaggacgtactgatgtcccgttgcatgtcgctaagctcc

SEQ ID NO: 10 - DNA, 40 bp, Artificial Sequence
  Synthetic (HFL aptamer)
cgcggacggggttttgtcccccgtagctgactaaatacat

SEQ ID NO: 11 - DNA, 40 bp, Artificial Sequence
  Synthetic (HFL aptamer)
acgggacgcgttctggtccctacttcttgtgtgtctattc

SEQ ID NO: 12 - DNA, 40 bp, Artificial Sequence
  Synthetic (HFL aptamer)
aatggacgcatcccagtcccccaagcttccgtatcgtaac

SEQ ID NO: 13 - DNA, 40 bp, Artificial Sequence
  Synthetic (HFL aptamer)
tgcggacgcacgcctgtcctcaacccatgattacacgttt

SEQ ID NO: 14 - DNA, 40 bp, Artificial Sequence
  Synthetic (HFL aptamer)
tggggacgggtatacgtccatctcgtatccaactaggcga

SEQ ID NO: 15 - DNA, 40 bp, Artificial Sequence
  Synthetic (HFL aptamer)
gtgggacgcgtgtaggtccaaacggtgcggtttggcgctc

SEQ ID NO: 16 - DNA, 40 bp, Artificial Sequence
  Synthetic (HFL aptamer)
ggaggacgagtggtcgtcccgctgggcaatagctcggggc

SEQ ID NO: 17 - DNA, 40 bp, Artificial Sequence
  Synthetic (HFL aptamer)
acaggacgccagagcgtccaaggtcgccgctagtagaccc

SEQ ID NO: 18 - DNA, 40 bp, Artificial Sequence
  Synthetic (HFL aptamer)
tccggacgtagcccggtcccagcgcacggtaccaccgtcg

SEQ ID NO: 19 - DNA, 40 bp, Artificial Sequence
  Synthetic (HFL aptamer)
ccaggacgactctaggtcctcatgagattagatatcggtt

SEQ ID NO: 20 - DNA, 40 bp, Artificial Sequence
  Synthetic (HFL aptamer)
gtcggacggaataacgtcctaaatatttatgcaacgggga

SEQ ID NO: 21 - DNA, 40 bp, Artificial Sequence
  Synthetic (Random aptamer)
cctcttgcgcgctccgtatcacatttatatgatggccagt

SEQ ID NO: 22 - DNA, 40 bp, Artificial Sequence
  Synthetic (Random aptamer)
tctcttacaacgggtcttggccataacacaataatcctgc

SEQ ID NO: 23 - DNA, 40 bp, Artificial Sequence
  Synthetic (Random aptamer)
cctaactcgaccagtgggctcggacggatgattaacccta

SEQ ID NO: 24 - DNA, 40 bp, Artificial Sequence
  Synthetic (Random aptamer)
cccctacatccctttttgccgcaaatcacccagctcctga

SEQ ID NO: 25 - DNA, 40 bp, Artificial Sequence
  Synthetic (Random aptamer)
cttccttgatcgctgcccgatttttggaataggttcctag

SEQ ID NO: 26 - DNA, 40 bp, Artificial Sequence
  Synthetic (Random aptamer)
ctcaggttgtcgatctaaccattggcgggtacgttgtacc

SEQ ID NO: 27 - DNA, 40 bp, Artificial Sequence
  Synthetic (Random aptamer)
cccatgtcagccggttaatcttaatgttgattcgagtcac

SEQ ID NO: 28 - DNA, 40 bp, Artificial Sequence
  Synthetic (Random aptamer)
ctcttgtcgcagtacctcaattgagcgttgtacacattgt

SEQ ID NO: 29 - DNA, 40 bp, Artificial Sequence
  Synthetic (Random aptamer)
tgtatttcagtaatccaacggcacttgtgacactgctact

SEQ ID NO: 30 - DNA, 40 bp, Artificial Sequence
  Synthetic (Random aptamer)
tactcagacgaatcaaatatcgtgtccaaagtcctccgag

SEQ ID NO: 31 - DNA, 40 bp, Artificial Sequence
  Synthetic (Random aptamer)
ccccctcaggattttccgtagcttgtctggcagcagctgt

SEQ ID NO: 32 - DNA, 40 bp, Artificial Sequence
  Synthetic (Random aptamer)
tggcatttaataaacaccctcgttaacgcgtgtgtggatt

SEQ ID NO: 33 - DNA, 40 bp, Artificial Sequence
  Synthetic (Random aptamer)
gcagctccattgaggctagaacgtgagtcgtaccccacca

SEQ ID NO: 34 - DNA, 40 bp, Artificial Sequence
  Synthetic (Random aptamer)
cgctattgggtaaaggatctctcgtagctctgcatgagcc

SEQ ID NO: 35 - DNA, 40 bp, Artificial Sequence
  Synthetic (Random aptamer)
gctggtgtggccgccggaagatgacttataccctgcactc

SEQ ID NO: 36 - DNA, 40 bp, Artificial Sequence
  Synthetic (Random aptamer)
cttttttccaaaccgggtgtgccacggcctgcgaatcggt

SEQ ID NO: 37 - DNA, 40 bp, Artificial Sequence
  Synthetic (Random aptamer)
tgggaagaaatcaatgaatgtctcactgatagctggtggt

SEQ ID NO: 38 - DNA, 40 bp, Artificial Sequence
  Synthetic (Random aptamer)
tcccgatttgatggggtgctgcttatgatagtcacgaaac

SEQ ID NO: 39 - DNA, 40 bp, Artificial Sequence
  Synthetic (Random aptamer)
aggcccgtttttacctactctcatatttggacaaccgggt

SEQ ID NO: 40 - DNA, 40 bp, Artificial Sequence
  Synthetic (Random aptamer)
ttatttgtccatacattatagcgcgacgtggaacaggtaa

SEQ ID NO: 41 - protein, 288 aa, Artificial Sequence
  Synthetic (TGFBRII ectodomain)
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLALAGSGSGHMHHHHHHSSGLVPRGSGMKQTAAAKFGRQHMDSPDLNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLEHHHHHH

SEQ ID NO: 42 - protein, 171 aa, Artificial Sequence
  Synthetic (VEGF ectodomain)
HHHHHHAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 43 - protein, 480 aa, Artificial Sequence
  Syntheric (CBF1)
MAWIKRKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTLEHHHHHH

SEQ ID NO: 44 - DNA, 40 bp, Artificial Sequence
  Synthetic (Nucleic acid construct)
  misc_feature (1)..(3): each n is A, T, G or C
  misc_feature (9)..(15): each n is A, T, G or C
  misc_feature (20)..(40): each n is A, T, G or C
nnnggacgnnnnnnngtccnnnnnnnnnnnnnnnnnnnnn

SEQ ID NO: 45 - RNA, 40 nt, Artificial Sequence
  Synthetic (Nucleic acid construct)
  misc_feature (1)..(3): each n is A, U, G or C
  misc_feature (9)..(15): each n is A, U, G or C
  misc_feature (20)..(40): each n is A, U, G or C
nnnggacgnnnnnnnguccnnnnnnnnnnnnnnnnnnnnn

SEQ ID NO: 46 - DNA, 86 bp, Artificial Sequence
  Synthetic (HFL Aptamer)
  misc_feature (24)..(26): each n is A, T, G or C
  misc_feature (32)..(38): each n is A, T, G or C
  misc_feature (43)..(63): each n is A, T, G or C
tagggaagagaaggacatatgatnnnggacgnnnnnnngtccnnnnnnnnnnnnnnnnnnnnnttgactagtacatgaccacttga

SEQ ID NO: 47 - DNA, 86 bp, Artificial Sequence
  Synthetic (HFL Aptamer)
  misc_feature (24)..(63): each n is A, T, G or C
tagggaagagaaggacatatgatnnnnnnnnnnnnnnnnnnnnnnnnnnnnnnnnnnnnnnnnttgactagtacatgaccacttga

(19) United States
(12) Patent Application Publication
(10) Pub. No.: US 2020/0332296 A1
(43) Pub. Date: Oct. 22, 2020

(54) APTAMER TEMPLATE AND METHOD FOR PREPARING APTAMER BY USING SAME

(71) Applicant: Nuclixbio, Seoul (KR)

(72) Inventors: Hoyoung KANG, Seoul (KR); Sunghak LEE, Gwangmyeong-si (KR); Dohyung KIM, Sejong-si (KR); Yewon YOUK, Incheon (KR)

(21) Appl. No.: 16/956,700

(22) PCT Filed: Dec. 28, 2018
(86) PCT No.: PCT/KR2018/016878

§ 371 Date: Jun. 22, 2020

(30) Foreign Application Priority Data
Dec. 28, 2017 (KR) 10-2017-0183057
Dec. 28, 2017 (KR) 10-2017-0183058
Sep. 21, 2018 (KR) 10-2018-0114416
Sep. 21, 2018 (KR) 10-2018-0114417

(51) Int. Cl. C12N 15/115; C12N 15/10

(52) U.S. Cl. CPC C12N 15/115; C12N 2310/16; C12N 2310/531; C12N 15/1006

(57) ABSTRACT

Provided are a novel nucleic acid structure, its use as an aptamer template, and a method for preparing an aptamer using thereof.

### TECHNICAL FIELD

The present invention relates to a novel nucleic acid structure, its use as an aptamer template, and a method for preparing an aptamer using thereof.

### BACKGROUND ART

Aptamers mean short length nucleic acid (RNA or DNA) molecules which bind to a certain target molecule with high affinity and specificity. Typical aptamers are about 20-80 nucleotides length, and the molecular weight is about 6-30 kDa. Aptamers form a particular tertiary structure, and by this structure, they can recognize a target molecule, and they bind to a target material by interaction similar to forming a complex by binding of antibody-antigen structurally. Due to such characteristics, aptamers have been paid attention as various molecular tools related to researches of therapy of diseases related to biosensors, drug screening, and the like or therapeutic materials themselves.

In addition, aptamers have competitive advantages superior to antibodies: At first, in aspect of immunogenicity, it has been demonstrated that aptamers show acceptable toxicity when administered into a body, while do not show immunogenicity. Since general immune responses do not recognize fragments of nucleic acids, it is very difficult to induce antibodies against aptamers. Second, in aspect of stability, aptamers have high resistance to relatively high temperatures (95° C. or higher,) and show high stability in circulation of normalization and subtraction processes. Furthermore, aptamers are easy to transfer in a lyophilized state, and can be stored at room temperature for a long period. Third, aptamers have an advantage capable of binding potentially to various and broad targets such as ion materials, organic or inorganic molecules, peptides, proteins, toxins, virus particles, whole cells, entire organs, living animals, and the like, in addition to nucleic acid molecules. Fourth, aptamers have an ability to distinguish two nucleic acid molecules that differ only by one nucleotide or conformational isomers. In addition, aptamers have convenience and availability of chemical modification, and they can be utilized by modification of various type, for example, sugar, backbone, base and other modification, and the like.

Nucleic acid-based medicines such as aptamers may be degraded by various enzymes in vivo such as nuclease, and the like when injected into a body, and therefore the pharmacological effect may be reduced. Due to such a reason, various modification for enhancing in vivo stability of nucleic acids has been researched. However, to provide modification for nucleic acids, additional costs are required, and also forms and physical properties of nucleic acids are affected all, and therefore, side effects such as increase of toxicity, degradation of binding ability to target molecules, and the like may be caused. In addition, the stability of the tertiary structure of aptamers is low, and thus when prepared as medicines, they have a structural disadvantage that the structure is unfolded at 37° C. which is the temperature in vivo and they may be under denaturation at the end.

As a general selection method of aptamers, ‘SELEX (Systematic Evolution of Ligands by Exponential Enrichment)’ has been widely used (See U.S. Pat. Nos. 5,475,096 and 5,270,163, the documents are incorporated herein by reference).

SELEX is a process of drawing aptamers targeting a specific molecule in an oligonucleotide library that is a large scale of random sequences. In SELEX, the oligonucleotide library is reacted with the target molecule, and sequences which do not bind to the target molecule are removed, and sequences which bind to the target molecule are separated and amplified through PCR (polymerase chain reaction), and then the aforementioned process is repeated many times, and finally, only the sequences which bind to the target molecule strongest are selected.

The typical SELEX process has several limitations. Any aptamer in the random sequences in the library may not bind to a target molecule strongly. In addition, a lot of time is taken in the SELEX process, and it requires shortly from a few days up to several months. Additional adjustment may be required to reduce the length of the sequence of the aptamer or provide biological properties, since the finally separated aptamer has a low functional ability. In addition, in case of a random sequence library generally used in the SELEX process, there is a high possibility that sequences which do not form a tertiary structure are included, and thus the screening efficiency may decrease.

Accordingly, there is a need to improve the process for avoiding such disadvantages of SELEX and selecting a more useful and effective aptamer sequence.

### DETAILED DESCRIPTION OF THE INVENTION

### Technical Problem

Herein, provided are a novel structure of nucleic acid structure and a technology for preparing an aptamer with excellent stability and binding ability to a target molecule, by using for aptamer selection using the same as an aptamer template.

One embodiment provides a nucleic acid structure represented by the following general formula 1:

5′-[T1][S1][L][S2][T2]-3′ (general formula 1)

wherein,

L site is a site forming a loop structure, and

S1 site and S2 site are sites forming a stem structure, and have anti-parallel complementary sequences (namely, the sequence from 5′ to 3′ direction of S¹ and the sequence from 3′ to 5′ direction of S2 are complementary each other), and

T1 site represents a 5′ end region and T2 represents a 3′ end region.

The nucleotide may be each independently selected from A, T/U (meaning T or U, same as below), G, and C.

In one embodiment, the nucleic acid structure may be represented by the following general formula 2:

5′-[(N)ₚ][(X)ₗ][G(N)ₙ][(X)ₘ][(N)_{q}]-3′ (general formula 2)

wherein,

N and X mean nucleotides selected from A, T/U, G, and C, and N is each independently selected from A, T/U, G, and C randomly, and X is each independently selected from A, T/U, G, and C randomly, and the ratio of C or G is 55% or higher (the upper limit is 100% or less than 100%) based on the total nucleotides of [(X)ₗ] or [(X)ₘ], and

[G(N)ₙ] is a site forming a loop structure, and n is the number of any nucleotide N, and may be an integer of 3 to 29, that is, 3, 4, 5, 6, 7, 8, 9, 10, 11, 12, 13, 14, 15, 16, 17, 18, 19, 20, 21, 22, 23, 24, 25, 26, 27, 28, or 29, and

[(X)ₗ] and [(X)ₘ] are sites forming a stem structure by forming a base pair with antiparallel complementary sequences each other, and l and m are numbers of any nucleotide X and each may be an integer of 3 to 10, for example, 3, 4, 5, 6, 7, 8, 9, or 10, and may be integers the same or different each other, for example, the same integers each other, and

[(N)ₚ] and [(N)_{q}] are 5′ end region and 3′ end region, respectively, and p and q are numbers of any nucleotide N, and each is independently an integer of 1 to 50 (integers the same or different each other), but not limited thereto.

In one embodiment, the nucleic acid structure may be represented by the following general formula 3, but not limited thereto:

(general formula 3; SEQ ID NO: 44)
NNNGGACGNN NNNNNGTCCN NNNNNNNNNN NNNNNNNNNN
(general formula 4; SEQ ID NO: 45)
NNNGGACGNN NNNNNGUCCN NNNNNNNNNN NNNNNNNNNN

(in the general formulas, N is each independently selected from A, T/U, G, and C, and the bolded part has anti-parallel complementary sequences and forms a stem structure, and the underlined part forms a loop structure)

The nucleic acid structure may further comprise a labeling material (fluorescent material, ligand (biotin, etc.)) and/or 1 to 30, 1 to 28, 1 to 25, 1 to 23, 1 to 20, 1 to 15, or 1 to 10 additional oligonucleotides (for example, may be a primer binding site) at the 5′ end and/or 3′ end, for amplification (for example, performing PCR), purification, and the like.

Other embodiment provides an aptamer template comprising the nucleic acid structure.

Other embodiment provides a composition for selecting or preparing a target material-specific binding nucleic acid molecule comprising the nucleic acid structure. The target material-specific binding nucleic acid molecule may be an aptamer.

Other embodiment provides a method for selecting or a method for preparing a target material-specific binding nucleic acid molecule using the nucleic acid structure.

The method for selecting or method for preparing may comprise

(1) contacting the nucleic acid structure with a target material; and

(2) measuring whether to bind between the nucleic acid structure and target material, and

may further comprise (3) determining the nucleic acid structure as a nucleic acid molecule specifically binding to the target material, when binding between the nucleic acid structure and target material is confirmed.

Other embodiment provides a method for selecting or a method for preparing a target material-specific binding nucleic acid aptamer using the nucleic acid structure.

The method for selecting or the method for preparing a nucleic acid aptamer may comprise

(a) contacting the nucleic acid structure with a target material; and

(b) measuring whether to bind between the nucleic acid structure and target material, and

may further comprise (c) determining the nucleic acid structure as a nucleic acid aptamer specifically binding to the target material, when binding between the nucleic acid structure and target material is confirmed.

In the methods, the nucleic acid structure used may be two or more kinds having different sequences each other at any nucleotide (N) position of general formulas 1 to 3.

The method for selecting or method for preparing a nucleic acid aptamer may be applied to the conventional SELEX method. In this case, the method for selecting or method for preparing a nucleic acid aptamer may comprise

(i) contacting a library comprising one or more kinds of the nucleic acid structures with beads in which a target material is immobilized;

(ii) washing the beads of the step (i); and

(iii) confirming the nucleic acid structure bound to the beads of the step (ii), and

may further comprise (iv) determining the nucleic acid structure confirmed in the step (iii) as a nucleic acid aptamer specifically binding to the target material.

Other embodiment provides a nucleic acid aptamer produced from the nucleic acid structure (aptamer template). Other embodiment provides a nucleic acid aptamer prepared by the method for preparing a nucleic acid aptamer. The nucleic acid aptamer may have the same nucleic sequences the nucleic acid structure and bind specifically to a target material.

### Technical Solution

Herein, it is suggested that the stability and binding ability to target molecules of aptamers can be enhanced by randomly providing a stem-loop structure at the starting region of the nucleic acid aptamer structure.

The technology provided herein relates to a technology of design or manufacturing of a nucleic acid aptamer, and more specifically, relates to a technology to allow selection of aptamers with excellent binding ability to target molecules and stability, by introducing a stem-loop structure to an aptamer library. In addition, the process of the technology provided herein is simple and can be easily performed, and therefore there is an advantage of low efforts and costs.

One embodiment provides a nucleic acid structure represented by the following general formula 1:

5′-[T1][S1][L][S2][T2]-3′ (general formula 1)

wherein,

L site is a site forming a loop structure, and may comprise 4 to 30, 4 to 28, 4 to 25, 4 to 23, 4 to 20, 4 to 18, 4 to 15, 4 to 13, 4 to 10, 5 to 30, 5 to 28, 5 to 25, 5 to 23, 5 to 20, 5 to 18, 5 to 15, 5 to 13, 5 to 10, 6 to 30, 6 to 28, 6 to 25, 6 to 23, 6 to 20, 6 to 18, 6 to 15, 6 to 13, 6 to 10, or about 8 nucleotides, and

S1 site and S2 site are sites forming a stem structure, and have anti-parallel complementary sequences (namely, the sequence from 5′ to 3′ direction of S¹ and the sequence from 3′ to 5′ direction of S2 are complementary each other) and may comprise 3 to 10, 3 to 8, 3 to 5, 4 to 10, 4 to 8, 4 to 5, or about 4 nucleotides, respectively, and S1 and S2 may comprise the same number of nucleotides, and

T1 site represents a 5′ end region (namely, extended site to the 5′ end of the S1 site forming a stem structure) and T2 site represents a 3′ end region (namely, extended site to the 3′ end of the S2 site forming a stem structure), and each independently, may comprise 1 to 50, 1 to 45, 1 to 40, 1 to 35, 1 to 30, 1 to 28, 1 to 25, 1 to 23, 1 to 20, 1 to 15, 1 to 10, 1 to 5, 2 to 50, 2 to 45, 2 to 40, 2 to 35, 2 to 30, 2 to 28, 2 to 25, 2 to 23, 2 to 20, 2 to 15, 2 to 10, 2 to 5, 3 to 50, 3 to 45, 3 to 40, 3 to 35, 3 to 30, 3 to 28, 3 to 25, 3 to 23, 3 to 20, 3 to 15, 3 to 10, 3 to 5, 5 to 50, 5 to 45, 5 to 40, 5 to 35, 5 to 30, 5 to 28, 5 to 25, 5 to 23, 5 to 20, 5 to 15, 5 to 10, 10 to 50, 10 to 45, 10 to 40, 10 to 35, 10 to 30, 10 to 28, 10 to 25, 10 to 23, 10 to 20, 10 to 15, 15 to 50, 15 to 45, 15 to 40, 15 to 35, 15 to 30, 15 to 28, 15 to 25, 15 to 23, 15 to 20, 17 to 50, 17 to 45, 17 to 40, 17 to 35, 17 to 30, 17 to 28, 17 to 25, 17 to 23, 17 to 20, 20 to 50, 20 to 45, 20 to 40, 20 to 35, 20 to 30, 20 to 28, 20 to 25, or 20 to 23 nucleotides, and T1 and T2 may comprise a different number of nucleotides, respectively.

The nucleotide may be each independently selected from A, T/U, G, and C.

In one embodiment, the L site may necessarily comprise guanine (G) at the 5′ end, and for example, it may be represented as follows:

5′-G(N)ₙ-3′ (N represents a nucleotide selected from A, G, C, and T/U(when 2 or more of nucleotides are comprised, they may be the same or different each other), n represents the number of nucleotides comprised in N, and it may be an integer of 3 to 29, that is, 3, 4, 5, 6, 7, 8, 9, 10, 11, 12, 13, 14, 15, 16, 17, 18, 19, 20, 21, 22, 23, 24, 25, 26, 27, 28, or 29, for example, 7, but not limited thereto). In one embodiment, the L site may have the sequence of 5′-GAAAAAAA-3′, but not limited thereto.

In one embodiment, the S¹ site and S2 site have anti-parallel complementary sequences (namely, the sequence from 5′ to 3′ direction of S¹ and the sequence from 3′ to 5′ direction of S2 are complementary each other) to form a stem structure, and form base pairs. Then, the binding ability of GC base pair is stronger than the binding ability of AT/U base pair, and therefore, the ratio of G or C sequence in the total sequence or the ratio of GC base pair of the total base pairs may be 55% or higher, 57% or higher, 60% or higher, 62% or higher, 66% or higher, 70% or higher, 71% or higher, 75% or higher, 77% or higher, 80% or higher, 83% or higher, 85% or higher, 87% or higher, or 88% or higher (the upper limit is 100% or less than 100%). Although not limited thereto, in order to avoid excessive presence of CG base pairs in the stem structure, the case where all nucleotides of the S¹ site and S2 site are C or G may be excluded herein. In other words, the S¹ site and S2 site may comprise at least one or more of A or T/U (but, it is not over 25%, 33% or 40% of the total S1 site and S2 site sequences), and then, A or T/U may be positioned 1, 2 or 3 nucleotides away from the L site, but not limited thereto. In one embodiment, the S¹ site may have the sequence of 5′-GGAC-3′ and the S2 site may have the sequence of 5′-GT/UCC-3′, but not limited thereto.

T1 site is the 5′ end region located adjacent to 5′ of S1 (extended site of 5′ end of S1), and may comprise 1 to 50, 1 to 45, 1 to 40, 1 to 35, 1 to 30, 1 to 28, 1 to 25, 1 to 23, 1 to 20, 1 to 15, 1 to 10, 1 to 5, 2 to 50, 2 to 45, 2 to 40, 2 to 35, 2 to 30, 2 to 28, 2 to 25, 2 to 23, 2 to 20, 2 to 15, 2 to 10, 2 to 5, 3 to 50, 3 to 45, 3 to 40, 3 to 35, 3 to 30, 3 to 28, 3 to 25, 3 to 23, 3 to 20, 3 to 15, 3 to 10, or 3 to 5 nucleotides the same or different each other. T2 site is the 3′ end region located adjacent to 3′ of S2 (extended site of 3′ end of S2), and may comprise 5 to 50, 5 to 45, 5 to 40, 5 to 35, 5 to 30, 5 to 28, 5 to 25, 5 to 23, 5 to 20, 5 to 15, 5 to 10, 10 to 50, 10 to 45, 10 to 40, 10 to 35, 10 to 30, 10 to 28, 10 to 25, 10 to 23, 10 to 20, 10 to 15, 15 to 50, 15 to 45, 15 to 40, 15 to 35, 15 to 30, 15 to 28, 15 to 25, 15 to 23, 15 to 20, 17 to 50, 17 to 45, 17 to 40, 17 to 35, 17 to 30, 17 to 28, 17 to 25, 17 to 23, 17 to 20, 20 to 50, 20 to 45, 20 to 40, 20 to 35, 20 to 30, 20 to 28, 20 to 25, or 20 to 23 nucleotides the same or different each other.

In one embodiment, the nucleic acid structure provided herein may have a structure in which a stem-loop structure is biased toward the 5′ end. In other words, in the nucleic acid structure, the number of nucleotides of T1 that is the extended site of the 5′ end of S1 in the stem structure may be less than the number of nucleotides of T2 that is the extended site of the 3′ end of S2 in the stem structure (T1 number<T2 number). For example, while satisfying the condition that the number of nucleotides of T2 exceeds the number of nucleotides of T¹, T1 may comprise 1 to 20, 1 to 15, 1 to 10, 1 to 5, 2 to 20, 2 to 15, 2 to 10, 2 to 5, 3 to 20, 3 to 15, 3 to 10, 3 to 5, 5 to 20, 5 to 15, 5 to 10, 10 to 20, 10 to 15, 15 to 20, or 4 nucleotides, and T2 may comprise 15 to 50, 15 to 45, 15 to 40, 15 to 35, 15 to 30, 15 to 28, 15 to 25, 15 to 23, 15 to 20, 17 to 50, 17 to 45, 17 to 40, 17 to 35, 17 to 30, 17 to 28, 17 to 25, 17 to 23, 17 to 20, 20 to 50, 20 to 45, 20 to 40, 20 to 35, 20 to 30, 20 to 28, 20 to 25, or 21 nucleotides.

In one embodiment, the nucleic acid structure may be represented by the following general formula 2:

5′-[(N)ₚ][(X)ₗ][G(N)ₙ][(X)ₘ][(N)_{q}]-3′ (general formula 2)

wherein,

N and X mean a nucleotide selected from A, T/U, G, and C, and N is each independently selected from A, T/U, G, and C randomly, and X is each independently selected from A, T/U, G, and C randomly, and the ratio of C or G is 55% or higher, 57% or higher, 60% or higher, 62% or higher, 66% or higher, 70% or higher, 71% or higher, 75% or higher, 77% or higher, 80% or higher, 83% or higher, 85% or higher, 87% or higher, or 88% or higher (the upper limit is 100% or less than 100%) based on the total nucleotides of [(X)ₗ] or [(X)ₘ], and

[G(N)ₙ] is a site forming a loop structure, and n is the number of any nucleotide N, and may be an integer of 3 to 29, that is, 3, 4, 5, 6, 7, 8, 9, 10, 11, 12, 13, 14, 15, 16, 17, 18, 19, 20, 21, 22, 23, 24, 25, 26, 27, 28, or 29, and

[(X)ₗ] and [(X)ₘ] are sites forming a stem structure by having anti-parallel complementary sequences to form a base pair, and 1 and m are the numbers of any nucleotide X, and they may be an integer of 3 to 10, for example, 3, 4, 5, 6, 7, 8, 9, or 10, respectively, and they may be an integer the same or different each other, for example, an integer the same each other, and

[(N)ₚ] and [(N)_{q}] are 5′ end region (extended site of the 5′ end of S1) and 3′ end region (extended site of the 3′ end of S2), respectively, and p and q are the numbers of any nucleotide N, and may be each independently an integer of 1 to 50 (may be an integer the same or different each other), for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50, respectively, and in one embodiment, p may be an integer of 1 to 30 (for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30), an integer of 1 to 20, an integer of 1 to 10, or an integer of 1 to 5, and q may be an integer of 10 to 50 (for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50), an integer of 15 to 50, or an integer of 20 to 50, but not limited thereto.

In one embodiment, p which is the number of nucleotides of the 5′ end region of S¹ (extended site of the 5′ end of S1) may be smaller than q which is the number of nucleotides of the 3′ end region of S2 (extended site of the 3′ end of S2). In one embodiment, while satisfying the condition of p<q, p may be an integer of 1 to 30 (for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30), and q may be an integer of 10 to 50 (for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50), but not limited thereto.

In one embodiment, the nucleic acid structure may be represented by the following general formula 3 or general formula 4, but not limited thereto:

(general formula 3; SEQ ID NO: 44)
NNNGGACGNN NNNNNGTCCN NNNNNNNNNN NNNNNNNNNN
(general formula 4; SEQ ID NO: 45)
NNNGGACGNN NNNNNGUCCN NNNNNNNNNN NNNNNNNNNN

(in the general formulas, N is each independently selected from A, T/U (T in the general formula 3, U in the general formula 4), G, and C, and the bolded part has anti-parallel complementary sequences and forms a stem structure, and the underlined part forms a loop structure)

The nucleic acid structure may further comprise a labeling material (fluorescent material, ligand (biotin, etc.)) and/or 1 to 30, 1 to 28, 1 to 25, 1 to 23, 1 to 20, 1 to 15, 1 to 10, 2 to 30, 2 to 28, 2 to 25, 2 to 23, 2 to 20, 2 to 15, 2 to 10, 3 to 30, 3 to 28, 3 to 25, 3 to 23, 3 to 20, 3 to 15, 3 to 10, 5 to 30, 5 to 28, 5 to 25, 5 to 23, 5 to 20, 5 to 15, or 5 to 10 additional oligonucleotides (for example, may be a primer binding site) at the 5′ end and/or 3′ end, for amplification (for example, performing PCR), purification, and the like.

The nucleic acid structure provided herein may be a DNA structure and/or an RNA structure. The nucleic acid structure may be a non-naturally occurring material, and may be chemically or recombinantly synthesized.

The nucleotide disclosed herein is represented as comprised bases, and T/U means T for the DNA structure and U for the RNA structure.

The nucleic acid structure may have a unique stem-loop secondary structure. The nucleic acid structure of stem-loop secondary structure may form a double strand at the stem site and may be a single strand form at other parts. In addition, the nucleic acid structure may form a library having various sequences by inserting any nucleotide to N of the general formula 2 or 3. The nucleic acid structure of stem-loop structure in which any nucleotide is inserted to N of the general formula 2 or 3 may be bound to a desired target material (for example, nucleic acid molecule (DNA, RNA, etc.), protein, peptide, and the like), and thereby it may be usefully applied for selecting an structure having excellent binding ability.

As described above, the nucleic acids structure or aptamer obtained therefrom provided herein, may retain one or more of stem-loop structures, and induce an increase of Tm value and/or decrease of free energy value, to secure excellent structural stability compared to the case without comprising a stem-loop structure. In addition, the nucleic acid structure and aptamer obtained therefrom can hinder degradation by nuclease such as exonuclease, and the like, by having a stem-loop structure biased toward the 5′ end, and therefore may secure excellent efficacy persistency in vivo (for example, in blood or in serum) and/or intracellular, compared to the nucleic acid structure not having such a structure (for example, the stem-loop structure is biased toward the center or 3′ end).

The target material used herein is a biomaterial to be targeted, and there is no special limitation, and it may be one or more kinds selected from the group consisting of nucleic acid molecules such as DNA, RNA, etc., peptides, proteins, small molecule compounds, and the like, and may be isolated from a living body or cell.

In one specific embodiment, the target material

may be a biomaterial with a pI (isoelectric point) value (for example, may be measured by IPC (Isoelectric Point Calculator)) of about 2 to about 14, about 2 to about 13, about 2 to about 12, about 2 to about 11, about 2 to about 10, about 2 to about 9.5, about 2 to about 9, about 2 to about 8.75, about 2 to about 8.5, about 3 to about 14, about 3 to about 13, about 3 to about 12, about 3 to about 11, about 3 to about 10, about 3 to about 9.5, about 3 to about 9, about 3 to about 8.75, about 3 to about 8.5, about 4 to about 14, about 4 to about 13, about 4 to about 12, about 4 to about 11, about 4 to about 10, about 4 to about 9.5, about 4 to about 9, about 4 to about 8.75, about 4 to about 8.5, about 4.5 to about 14, about 4.5 to about 13, about 4.5 to about 12, about 4.5 to about 11, about 4.5 to about 10, about 4.5 to about 9.5, about 4.5 to about 9, about 4.5 to about 8.75, about 4.5 to about 8.5, about 5 to about 14, about 5 to about 13, about 5 to about 12, about 5 to about 11, about 5 to about 10, about 5 to about 9.5, about 5 to about 9, about 5 to about 8.75, about 5 to about 8.5, about 5.5 to about 14, about 5.5 to about 13, about 5.5 to about 12, about 5.5 to about 11, about 5.5 to about 10, about 5.5 to about 9.5, about 5.5 to about 9, about 5.5 to about 8.75, or about 5.5 to about 8.5, for example, a polypeptide comprising 2 or more of amino acids. When the target material is a polypeptide (for example, human protein), the polypeptide having a pI value in the above range may be confirmed through a well-known route (for example, see http://isoelectricpointdb.org/40/UP000005640_9606_all_isoelectric_point_proteome_Homo_sapiens_Human.html). In general, it is common that the lower the pI value of the target material is, the weaker binding with aptamers is due to electrostatic repulsive power, but the aptamer selected using the nucleic acid structure provided herein has an advantage of high binding ability even for materials with a relatively low pI value (for example, proteins with a pI value of 7 or less, 6.8 or less, 6.5 or less, 6.2 or less, 6 or less, or 5.8 or less).

The target material may be a biomaterial having a weight average molecular weight of about 1 to about 1000 KDa, about 1 to about 500 KDa, about 1 to about 300 KDa, about 1 to about 100 KDa, about 1 to about 100 KDa, about 1 to about 90 KDa, about 1 to about 80 KDa, about 1 to about 70 KDa, about 1 to about 60 KDa, about 10 to about 1000 KDa, about 10 to about 500 KDa, about 10 to about 300 KDa, about 10 to about 100 KDa, about 10 to about 90 KDa, about 10 to about 80 KDa, about 10 to about 70 KDa, about 10 to about 60 KDa, about 15 to about 1000 KDa, about 15 to about 500 KDa, about 15 to about 300 KDa, about 15 to about 100 KDa, about 15 to about 90 KDa, about 15 to about 80 KDa, about 15 to about 70 KDa, or about 15 to about 60 KDa, for example, polypeptide but not limited thereto. For example, the target material may be one or more kinds selected from all polypeptides (two or more amino acids linked by peptide bonds; for example, proteins and/or peptides) which can act as a marker of physiological and/or pathological phenomena or show activity in a living body. For example, the target material may be one or more kinds selected from the group consisting of enzymes, hormones, growth factors, receptors, transport polypeptides, immune polypeptides (collectively refer to polypeptides made in immunocytes), signaling polypeptides, polypeptides composing living body, toxoproteins, immunogens of bacteria or viruses (surface protein antigens, etc.), and the like.

In one specific embodiment, the target material may be one or more kinds selected from the group consisting of

enzymes including hydrolase (for example, protease, phosphatase, etc.), oxidoreductase, transferase of uridine, methyl group, phosphate group (for example, uridylyl transferase (for example, Terminal Uridylyltransferase 7, etc.), kinase, etc.), and the like,

hormones including insulin, growth hormone, growth hormone releasing hormone, melatonin, serotonin, thyroid hormone, thyroid stimulating hormone, thyroid stimulating hormone releasing hormone, epinephrine, norepinephrine, dopamine, adiponectin, adrenocorticotropic hormone, adrenocorticotropic hormone releasing hormone, vasopressin, calcitonin, cholecystokinin, follicle stimulating hormone, gastrin, ghrelin, glucagon, human chorionic gonadotropin, luteinizing hormone, parathormone, prolactin, secretin, lipotropin, histamine, and the like,

growth factors including insulin-Like growth factors (IGFs), epidermal growth factor (EGF), VEGF(Vascular endothelial growth factor), Angiopoietin, nerve growth factor (NGF), Erythropoietin (EPO), Fibroblast growth factor (FGF), Platelet-derived growth factor (PDGF), Transforming growth factor (TGF), and the like,

receptors including G protein-coupled receptor (GPCR), receptor tyrosine kinase (RTK), ionotropic receptor, and the like,

transport polypeptides such as hemoglobin, transferrin, and the like,

immune polypeptides including immunoglobulin (for example, IgG (IgG1, IgG2, IgG3, IgG4), IgA, IgD, IgM IgE, etc.), cytokine (for example, interleukin such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18), tumor necrosis factor (TNF) such as TNF-alpha, beta or gamma, interferon(IFN)-alpha, -beta, -gamma, -omegaor-tau, TRAIL(TNF-related apoptosis-inducing ligand), colony stimulating factor (CSF) (for example, G-CSF(Granulocyte-colony stimulating factor), GM-CSF(Granulocyte-macrophage colony-stimulating factor), M-CSF(macrophage colony-stimulating factor), etc.), and the like,

various kinds of signaling polypeptides such as extracellular matrix glycoprotein (for example, Reelin, etc.), Bone morphogenetic protein (BMP), and the like,

except for that, polypeptides composing living body such as collagen, elastin, keratin, tubulin, actin, fibrin, myosin, albumin, histone, casein, ovalbumin, and the like,

toxoproteins such as diphtheria, and the like,

surface protein antigens of viruses (for example, corona virus (e.g., MERS (Middle East respiratory syndrome) corona virus, etc.), influenza virus (for example, influenza type A (H1N1, H1N2, H2N2, H2N3, H3N1, H3N2, H3N8, H5N1, H5N2, H5N3, H5N6, H5N8, H5N9, H7N1, H7N2, H7N3, H7N4, H7N7, H7N9, H9N2, H10N7, etc.), influenza type B, influenza type C, etc.), measles virus, mumps virus, rubella virus, poxvirus, yellow fever virus, encephalitis virus, rabies virus, hepatitis A virus, hepatitis B virus, epidemic hemorrhagic fever virus, etc.) or bacteria (for example, tubercular bacillus, haemophilus pertussis, typhoid bacillus, cholera bacillus, pneumococcus, Clostridium tetani, etc.), and

transcription factors (TF) (for example, TATA box binding protein (TBP) such as TFIIA, TFIIB, TFIID, TFIIE, TFIIF, TFIIH, TFIIJ, etc., TBP associated factor (TAF), etc.), and the like.

Other embodiment provides an aptamer template comprising the nucleic acid structure. Other embodiment provides a composition for selecting or preparing a target material-specific binding nucleic acid molecule comprising the nucleic acid structure. The target material-specific binding nucleic acid molecule may be an aptamer.

Other embodiment provides a method for selecting or a method for preparing a target material-specific binding nucleic acid molecule using the nucleic acid structure.

The method for selecting or method for preparing may comprise

(1) contacting the nucleic acid structure with a target material; and

(2) measuring whether to bind between the nucleic acid structure and target material, and

may further comprise

(3) determining the nucleic acid structure as a nucleic acid molecule specifically binding to the target material, when binding between the nucleic acid structure and target material is confirmed.

Other embodiment provides a method for selecting or a method for preparing a nucleic acid aptamer specifically binding to a target material using the nucleic acid structure.

The method for selecting or method forpreparing a nucleic acid aptamer may comprise

(a) contacting the nucleic acid structure with a target material; and

(b) measuring whether to bind between the nucleic acid structure and target material, and

may further comprise

(c) determining the nucleic acid structure as a nucleic acid aptamer specifically binding to the target material, when binding between the nucleic acid structure and target material is confirmed.

In the methods, the used nucleic acid structure may be 2 or more kinds having a sequence different each other at the position of any nucleotide (N) of general formulas 1 to 3.

The method for selecting or method for preparing a nucleic acid aptamer may be applied for conventional SELEX method. In this case, the method for selecting or method for preparing a nucleic acid aptamer may comprise

(i) contacting a library comprising one or more kinds of nucleic acid structures with beads in which a target material is immobilized;

(ii) washing the beads of the step (i); and

(iii) confirming the nucleic acid structure bound to the beads of the step (ii), and

may further comprise

(iv) determining the nucleic acid structure confirmed in the step (iii) as a nucleic acid aptamer specifically binding to the target material.

The method provided herein may be performed in vitro or in vivo, and for example, may be performed in vitro.

Other embodiment provides a nucleic acid aptamer produced from the nucleic acid structure (aptamer template). Other embodiment provides a nucleic acid aptamer prepared by the method for preparing a nucleic acid aptamer. As used herein, the aptamer template may mean a production frame in which certain nucleotide is inserted to any nucleotide (N position) to select and/or prepare a desired aptamer. Accordingly, the nucleic acid aptamer may have the same nucleic sequences the nucleic acid structure (aptamer template) and be capable of binding to a target material specifically.

In one embodiment, the nucleic acid structure has an advantage of excellent binding ability to a target material and/or stability of a prepared aptamer, without comprising a modified nucleotide as any nucleotide, which is modified for enhancing binding ability to a target material and/or enhancing stability of an aptamer. In other embodiment, the nucleic acid structure may comprise a modified nucleotide to prepare an aptamer with further enhanced stability and/or binding ability. The modified nucleotide may be one or more kinds selected from the group consisting of 2′-position sugar modification, 5′-position pyrimidine modification (for example, 5-(N-benzylcarboxyamide)-2′-deoxyuridine, 5-(N-isobutylcarboxyamide)-2′-deoxyuridine, 5-(N-tryptaminocarboxylamide)-2′-deoxyuridine, 5-(N41-(3-trimethylammonium)propyllcarboxyamide)-2′-deoxyuridine chloride, 5-(N-naphthylmethylcarboxyamide)-2′-deoxyuridine, or 5-(N41-(2,3-dihydroxypropyllcarboxyamide)-2′-deoxyuridine), modification in exocyclic amines, substitution of 4-thiouridine, substitution of 5-bromo- or 5-iodo-uracil, backbone modification, methylation, and unusual base-pairing combinations such as isobasesisocytidine and isoguanidine, etc., but not limited thereto.

In one specific embodiment, by composing a stem part (S¹ and S2 sites) of a stem-loop structure with 4 base pairs, a desired secondary structure (stem-loop structure) may be maintained even over the body temperature range (about 37° C.) (See Example 1-3). Then, for the base pairs forming the stem part, GC binding is more stable than AT/U binding, but AT base pair was inserted in the third stem part in order to avoid excessively high GC ratio. The loop part except for stem is a random sequence, but the sequence in which the loop starts is set to G, because designating G at this position further stabilizes the stem-loop structure.

### Advantageous Effects

The technology provided herein relates to a Nucleic Acid Aptamer design technology, and it can be usefully applied for preparing an aptamer with enhanced binding ability to a target molecule and strengthened stability, using an aptamer library comprising nucleic acid structures in which a stem-loop structure is introduced. In addition, this technology is simple and technically easy, and also has an advantage of being able to prepare an aptamer at a low cost.

### BRIEF DESCRIPTION OF THE DRAWINGS

FIG. 1 schematically shows the structure of the aptamer template according to one example.

FIG. 2 is a graph showing the Tm value of the aptamer obtained by using the aptamer template (HFL) according to one example, by comparing with the Tm value of the aptamer obtained by using a random library.

FIG. 3 is a graph showing the free energy (AG) of the aptamer obtained by using the aptamer template (HFL) according to one example, by comparing with the free energy (AG) of the aptamer obtained by using a random library.

FIG. 4 is a graph showing binding of the VEGF-specific aptamer obtained by using the aptamer template (HFL) according to one example to VEGF (measured by the degree of luminescence) as a fold of the background value.

FIG. 5 is a graph showing binding of the CBF1-specific aptamer obtained by using the aptamer template (HFL) according to one example to CBF1 (measured by the degree of luminescence) as a fold of the background value.

FIG. 6 is a graph showing binding of the TGFBRII ectodomain-specific aptamer obtained by using the aptamer template (HFL) according to one example to TGFBRII ectodomain (measured by the degree of luminescence) as a fold of the background value.

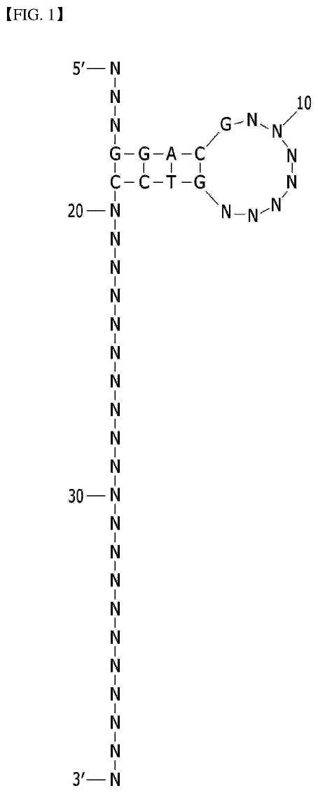

### MODE FOR CARRYING OUT THE INVENTION

Hereinafter, the present invention will be described in more detail by examples, but they are illustrative and are not intended to limit the scope of the present invention. It is apparent to those skilled in the art that the examples described below can be modified without departing from the essential gist of the invention.

### Example 1: Aptamer library production

Based on a aptamer template of stem-loop structure having the following general formula, any base was introduced to N position to prepare a DNA aptamer library with a total length of 86mer (hereinafter, ‘HFL (High Functional Library)’):

(general formula 5; SEQ ID NO: 46)
5′-TAG GGA AGA GAA GGA CAT ATG AT
[NNNGGACGNN NNNNNGTCCN NNNNNNNNNN NNNNNNNNNN]
TTG ACT AGT ACA TGA CCA CTT GA-3′

(in the general formula, N is each independently selected from A, T, G, and C, and the bolded part has anti-parallel complementary sequences and forms a stem structure, and the underlined part forms a loop structure; the part indicated in italics is an added sequence that can bind to forward and reverse primers).

For comparison, an oligonucleotide having the following general formula (86mer in total; hereinafter, ‘Random library’) was prepared:

(SEQ ID NO: 47
5′-TAG GGA AGA GAA GGA CAT ATG AT [N]₄₀
TTG ACT AGT ACA TGA CCA CTT GA-3′;

N is each independently selected from A, T, G, and C, and is different from the sequence in [ ] of the general formula 5)

All oligomers were produced in a form of Biotinylated-ssDNA(86mer) by commissioning to Bioneer corporation.

### Example 2: DNA Aptamer Analysis

For the ssDNA (aptamer) library prepared using HFL and Random library obtained in Example 1, PCR was performed using the following primers.

Forward primer:
5′-biotin-TAG GGA AGA GAA GGA CAT ATG AT 3′ (23mer)
Reverse primer:
5′-TCA AGT GGT CAT GTA CTA GTC AA-3′ (23mer)

The PCR process is summarized as follows:

(1) PCR mixture: Reverse primer(10 uM), forward primer(10 uM), dNTP(each 2 mM), pfu(+) DNA buffer (CANCERROP Co., Ltd.), pfu(+) DNA polymerase(1 U; CANCERROP Co., Ltd.), and Library prepared in Example 1 (100 ng).

(2) First cycles: 95° C. for 3 min, 53° C. for 30 sec, 72° C. for 30 min, 4° C. hold.

(3) add the forward primer and mix.

(4) Second cycles: 95° C. for 2 min, start cycle (4 cycles)—95° C. for 30 sec, 53° C. for 30 sec, 72° C. for 10 sec-end cycle, 72° C. for 5 min, 4° C. hold.

For the obtained PCR products, cloning was performed using ToPcloner PCR Cloning kit (enzynomics Co., Ltd.). For cloning, pTOP Blunt V2(10 ng) 1 ul, TOPcloner buffer (1.2M NaCl, 0.06M MgCl₂) 1 ul, and PCR product(4 ul) were mixed, and then it was left for 5 minutes at room temperature to prepare cloning product 6 ul.

Using the prepared cloning product, DH5a chemically competent E. coli was transformed. More specifically, cloning product 6 ul and DH5a chemically competent E. coli (CANCERROP Co., Ltd.) 5×10⁷ were mixed, and it was left in ice for 30 minutes and left at 42° C. for 2 minutes, and then it was immediately put in ice. After that, the cells were put in SOC media (2.0% tryptone, 0.5% yeast extract, 10 mM NaCl, 2.5 mM KCl, 10 mM MgCl2, 10 mM MgSO4, 20 mM Glucose) and mixed, and then they were left at 37° C. for 1 hour, and then were under spin down at 5000×g for 2 minutes, and the supernatant was removed, and then SOC media 200 ul was added again and mixed. The 5×10⁷ prepared cells were spread on 90 π LB plate (+Ampicillin 50 ug/ml) and then were cultured at 37° C. overnight. The colony was picked and it was cultured at 37° C. in a shaking incubator overnight.

To extract plasmid DNA in the cultured cells, Mini-prep was performed using mini-prep kit (CANCERROP Co., Ltd.). More specifically, the cell culture was centrifuged at 13000×g for 2 minutes to collect cells. The supernatant was removed and 51 buffer 250 ul of mini-prep kit (CANCERROP Co., Ltd.) was added and mixed well, and then S2 buffer 250 ul was added, and 4-6 times inverts were performed and S3 buffer 350 ul was added and 6 times inverts were performed, and then it was centrifuged at 13000×g for 10 minutes and only the supernatant was collected and was loaded in a column, and then it was centrifuged at 13000 rpm for 1 minute. PW buffer (CANCERROP Co., Ltd.) 700 ul was loaded in a column, and then it was centrifuged at 13000 rpm for 1 minutes twice, and then the column was put in a new tube, and D.W 50 ul was loaded in the center of the column membrane, and it was left for 2 minutes, and then it was centrifuged at 13000 rpm for 1 minute.

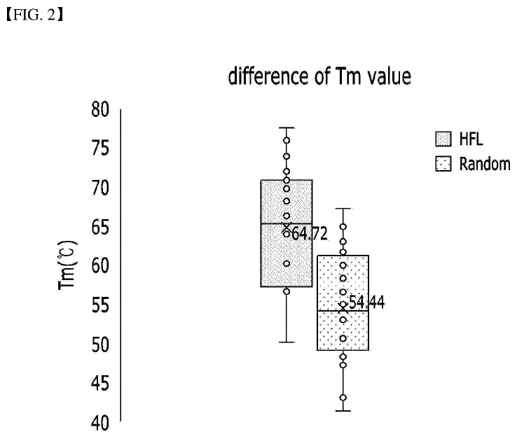

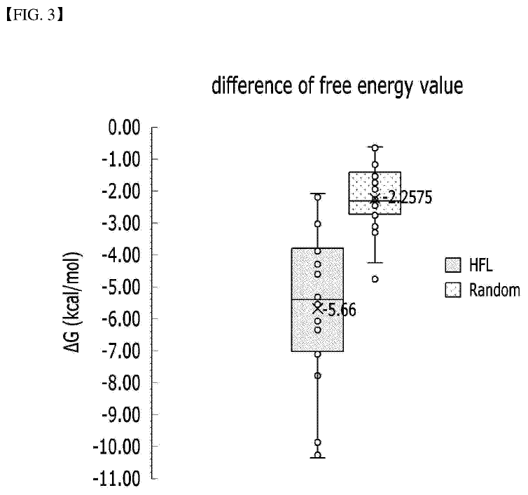

The sequence of DNA (20 aptamer DNAs selected in HFL and 20 aptamer DNAs obtained using Random library) obtained by the above method was analyzed (commissioned to Macrogen Co., Ltd.; reading the sequence with Universal M13 forward primer). In addition, free energy value and the Tm value of the aptamer section were obtained through ‘Mfold’ program (http://unafold.rna.albany.edu/?q=mfold) and the result of comparing the average value was shown in the following Tables 1 and 2, and FIG. 2 and FIG. 3.

TABLE 1
Free energy(ΔG), Tm, and sequence
of aptamers obtained using HFL.

| | SEQ | | |
|---|---|---|---|
| | ID | Tm | ΔG |
| sequence | NO. | (° C.) | (kcal/mol) |
| ATAGGACGGATGAGCGTCCG | 1 | 77.3 | −10.29 |
| GCCGGATGGGTTTTCCATCA | | | |
| CTGGGACGCGGATGAGTCCG | 2 | 66.2 | −6.35 |
| CTCCACCAGCCATCCATGTA | | | |
| TTAGGACGCGAAGATGTCCT | 3 | 60.1 | −5.34 |
| TGCAAGGGCTCATCGCATCA | | | |
| ATGGGACGACCCTGTGTCCC | 4 | 57.1 | −5.48 |
| TATATGCGCGGAGAGCCATC | | | |
| CTTGGACGCCCTCGTGTCCC | 5 | 57.1 | −4.30 |
| TTGCTCCTTCACATACGCAA | | | |
| GCTGGACGTTCGTGGGTCCC | 6 | 56.8 | −2.10 |
| ATTCTGAAGCCTAAGACTGG | | | |
| GAGGGACGAACAAGCGTCCA | 7 | 64.2 | −6.07 |
| GCGGTTATGCACCTTCGGTA | | | |
| TACGGACGCACCTCCGTCCG | 8 | 75.8 | −10.33 |
| GTTCTCAGCGTGGGGCGCAC | | | |
| GCAGGACGTACTGATGTCCC | 9 | 57.0 | −3.74 |
| GTTGCATGTCGCTAAGCTCC | | | |
| CGCGGACGGGGTTTTGTCCC | 10 | 70.8 | −6.93 |
| CCGTAGCTGACTAAATACAT | | | |
| ACGGGACGCGTTCTGGTCCC | 11 | 66.5 | −3.88 |
| TACTTCTTGTGTGTCTATTC | | | |
| AATGGACGCATCCCAGTCCC | 12 | 56.5 | −2.20 |
| CCAAGCTTCCGTATCGTAAC | | | |
| TGCGGACGCACGCCTGTCCT | 13 | 50.1 | −3.05 |
| CAACCCATGATTACACGTTT | | | |
| TGGGGACGGGTATACGTCCA | 14 | 63.9 | −5.59 |
| TCTCGTATCCAACTAGGCGA | | | |
| GTGGGACGCGTGTAGGTCCA | 15 | 60.7 | −5.20 |
| AACGGTGCGGTTTGGCGCTC | | | |
| GGAGGACGAGTGGTCGTCCC | 16 | 70.8 | −7.77 |
| GCTGGGCAATAGCTCGGGGC | | | |
| ACAGGACGCCAGAGCGTCCA | 17 | 73.8 | −9.86 |
| AGGTCGCCGCTAGTAGACCC | | | |
| TCCGGACGTAGCCCGGTCCC | 18 | 71.9 | −7.08 |
| AGCGCACGGTACCACCGTCG | | | |
| CCAGGACGACTCTAGGTCCT | 19 | 68.1 | −3.02 |
| CATGAGATTAGATATCGGTT | | | |
| GTCGGACGGAATAACGTCCT | 20 | 69.7 | −4.61 |
| AAATATTTATGCAACGGGGA | | | |
| average | | 64.72 | −5.66 |

TABLE 2
Free energy(ΔG), Tm, and sequence of aptamers
obtained using Random library.

| | SEQ | | |
|---|---|---|---|
| | ID | Tm | ΔG |
| sequence | NO. | (° C.) | (kcal/mol) |
| CCTCTTGCGCGCTCCGTATC | 21 | 47.1 | −1.55 |
| ACATTTATATGATGGCCAGT | | | |
| TCTCTTACAACGGGTCTTGG | 22 | 50.9 | −1.11 |
| CCATAACACAATAATCCTGC | | | |
| CCTAACTCGACCAGTGGGCT | 23 | 48.2 | −1.19 |
| ACGGACGGATGTTAACCCTA | | | |
| CCCCTACATCCCTTTTTGCC | 24 | 41.3 | −0.67 |
| GCAAATCACCCAGCTCCTGA | | | |
| CTTCCTTGATCGCTGCCCGA | 25 | 61.6 | −4.78 |
| TTTTTGGAATAGGTTCCTAG | | | |
| CTCAGGTTGTCGATCTAACC | | | |
| ATTGGCGGGTACGTTGTACC | 26 | 56.4 | −4.24 |
| CCCATGTCAGCCGGTTAATC | 27 | 43.0 | −0.63 |
| TTAATGTTGATTCGAGTCAC | | | |
| CTCTTGTCGCAGTACCTCAA | 28 | 55.1 | −1.74 |
| TTGAGCGTTGTACACATTGT | | | |
| TGTATTTCAGTAATCCAACG | 29 | 58.2 | −1.39 |
| GCACTTGTGACACTGCTACT | | | |
| TACTCAGACGAATCAAATAT | 30 | 62.9 | −2.38 |
| CGTGTCCAAAGTCCTCCGAG | | | |
| CCCCCTCAGGATTTTCCGTA | 31 | 52.9 | −2.79 |
| GCTTGTCTGGCAGCAGCTGT | | | |
| TGGCATTTAATAAACACCCT | 32 | 50.5 | −1.96 |
| CGTTAACGCGTGTGTGGATT | | | |
| GCAGCTCCATTGAGGCTAGA | 33 | 59.8 | −3.12 |
| ACGTGAGTCGTACCCCACCA | | | |
| CGCTATTGGGTAAAGGATCT | 34 | 67.1 | −3.31 |
| CTCGTAGCTCTGCATGAGCC | | | |
| GCTGGTGTGGCCGCCGGAAG | 35 | 61.6 | −2.43 |
| ATGACTTATACCCTGCACTC | | | |
| CTTTTTTCCAAACCGGGTGT | 36 | 53.3 | −2.28 |
| GCCACGGCCTGCGAATCGGT | | | |
| TGGGAAGAAATCAATGAATG | 37 | 64.9 | −2.67 |
| TCTCACTGATAGCTGGTGGT | | | |
| TCCCGATTTGATGGGGTGCT | 38 | 54.7 | −1.76 |
| GCTTATGATAGTCACGAAAC | | | |
| AGGCCCGTTTTTACCTACTC | 39 | 48.4 | −2.46 |
| TCATATTTGGACAACCGGGT | | | |
| TTATTTGTCCATACATTATA | 40 | 50.9 | −2.69 |
| GCGCGACGTGGAACAGGTAA | | | |
| average | | 54.44 | −2.26 |

As shown in Tables 1 and 2, and FIG. 2 and FIG. 3, the aptamer obtained from the aptamer template having a stem-loop structure of the present invention (HFL), was confirmed to have higher Tm value and lower free energy value, compared to the aptamer obtained from the conventional random library, and this result shows that an aptamer with enhanced stability can be prepared without an additional stabilization process, using the aptamer template provided herein.

### Example 3: Protein SELEX Using Library—TGFBRII Ectodomain, VEGF, & CBF1

pI value means the pH at which the net charge of protein becomes ‘0’, and the value is determined by kinds of amino acids composing protein. Since an acidic amino acid has one more carboxyl group (—COOH) and a basic amino acid has one more amino group (—NH2), the more the acidic amino acid is in protein, the smaller the pI value is, and the more the basic amino acid is, the bigger the pI value is. When specific protein is present in a higher pH environment (for example, buffer) than its pI value, the protein has a negative charge (−), and in the lower pH environment (for example, buffer), it has a positive charge (+).

In the following example, three proteins belonging to different pI ranges (acidic/basic/hydrophobic), respectively, in the buffer environment of physiological pH 7.5 of human were selected to progress an experiment, and thereby it was confirmed that the HFL library of the present invention had high binding affinity to target protein than Random library in all different charge environments.

More specifically, using the DNA aptamer library prepared in Example 1 (Biotinylated-ssDNA (86mer) library), SELEX (Systematic evolution of ligands by exponential enrichment) was performed for the following three proteins:

TGFBRII ectodomain: N terminal 6×His-tagged, C terminal Thioredoxin-tagged, 35 KDa, pi value: 5.74

(MSDKIIHLTDDSFDTDVLKADGAILVDFWAEWCGPCKMIAPILDEIA
DEYQGKLTVAKLNIDQNPGTAPKYGIRGIPTLLLFKNGEVAATKVGAL
SKGQLKEFLDANLALAGSGSGHMHHHHHHSSGLVPRGSGMKQTAAAKF
GRQHMDSPDLNDMIVTDNNGAVKFPQLCKFCDVRFSTCDNQKSCMSNC
SITSICEKPQEVCVAVWRKNDENITLETVCHDPKLPYHDFILEDAASP
KCIMKEKKKPGETFFMCSCSSDECNDNIIFSEEYNTSNPDLLEHHHH
HH; SEQ ID NO: 41);

VEGF: AbcamCo., Ltd., N terminal 6×His-tagged VEGFA protein, 22 KDa, pi value:

7.6

(HHHHHHAPMA EGGGQNHHEV VKFMDVYQRS YCHPIETLVD
IFQEYPDEIE YIFKPSCVPL MRCGGCCNDE GLECVPTEES
NITMQIMRIK PHQGQHIGEM SFLQHNKCEC RPKKDRARQE
NPCGPCSERR KHLFVQDPQT CKCSCKNTDS RCKARQLELN
ERTCRCDKPR R; SEQ ID NO: 42);

CBF1: C terminal 6×His-tagged, 54 KDa, pi value: 8.3

(MAWIKRKFGE RPPPKRLTRE AMRNYLKERG DQTVLILHAK
VAQKSYGNEK RFFCPPPCVY LMGSGWKKKK EQMERDGCSE
QESQPCAFIG IGNSDQEMQQ LNLEGKNYCT AKTLYISDSD
KRKHFMLSVK MFYGNSDDIG VFLSKRIKVI SKPSKKKQSL
KNADLCIASG TKVALFNRLR SQTVSTRYLH VEGGNFHASS
QQWGAFFIHL LDDDESEGEE FTVRDGYIHY GQTVKLVCSV
TGMALPRLII RKVDKQTALL DADDPVSQLH KCAFYLKDTE
RMYLCLSQER IIQFQATPCP KEPNKEMIND GASWTIISTD
KAEYTFYEGM GPVLAPVTPV PVVESLQLNG GGDVAMLELT
GQNFTPNLRV WFGDVEAETM YRCGESMLCV VPDISAFREG
WRWVRQPVQV PVTLVRNDGI IYSTSLTFTY TPEPGPRPHC
SAAGAILRAN SSQVPPNESN TNSEGSYTNA STLEHHHHHH;
SEQ ID NO: 43).

Protein SELEX was progressed in 3 rounds for each protein according to the scheme of the following Tables 3 to 5:

TABLE 3

| | ssDNA (DNA aptamer) | | TGFBRII | | Molar Ratio |
|---|---|---|---|---|---|
| Round | pM | μg | pM | μg | ssDNA : protein |
| round 1 | 500 | 13.95 | 50 | 1.75 | 10 : 1 |
| round 2 | 100 | 2.8 | 10 | 0.35 | 10 : 1 |
| round 3 | 100 | 2.8 | 5 | 0.18 | 20 : 1 |

TABLE 4

| | ssDNA (DNA aptamer) | | VEGF | | Molar Ratio |
|---|---|---|---|---|---|
| Round | pM | μg | pM | μg | ssDNA : protein |
| round 1 | 500 | 13.95 | 50 | 1.1 | 10 : 1 |
| round 2 | 100 | 2.8 | 10 | 0.22 | 10 : 1 |
| round 3 | 100 | 2.8 | 5 | 0.11 | 20 : 1 |

TABLE 5

| | ssDNA (DNA aptamer) | | CBF1 | | Molar Ratio |
|---|---|---|---|---|---|
| Round | pM | μg | pM | μg | ssDNA : protein |
| round 1 | 500 | 13.95 | 50 | 2.7 | 10 : 1 |
| round 2 | 100 | 2.8 | 10 | 0.54 | 10 : 1 |
| round 3 | 100 | 2.8 | 5 | 0.27 | 20 : 1 |

Ni-NTA magnetic bead 20 ul of Qiagen Co., Ltd. was transferred to an E-tube, and the supernatant was removed using a magnetic rack. Using SELEX buffer (P B/W buffer) 100 ul, beads were washed three times. The composition of SELEX buffer used for each protein herein was as follows: TGFBRII SELEX buffer (P B/W buffer): 12.5 mM Tris-HCl (pH 7.5), 125 mM NaCl, 1 mM KCl, 1 mM MgCl2, 1 mM PMSF (phenylmethanesulfonylfluoride or phenylmethylsulfonyl fluoride);

VEGF SELEX buffer (P B/W buffer): 12.5 mM Tris-HCl(pH 7.5), 125 mM NaCl, 0.25 mM DTT(dithiothreitol), 50 mM KCl,

CBF1 SELEX buffer (P B/W buffer): 12.5 mM Tris-HCl(pH 7.5), 125 mM NaCl, 50 mM KCl, 1 mM DTT (dithiothreitol).

Then, the protein prepared in advance (TGFBRII ectodomain, VEGF, or CBF1) was added in an amount of 50 pM to the SELEX buffer 100 ul, and then it was under binding to beads at 4° C. for 1 hour. After completing protein binding, the supernatant was removed, and beads were washed three times using SELEX buffer 100 ul. Then, after adding SELEX buffer 100 ul and biotinylated-ssDNA library 500 pM prepared in Example 1 to the protein-bound Ni-NTA beads, it was reacted at 37° C. for 1 hour, to bind DNA library (Example 1; HFL library and Random library) to protein-beads (DNA:protein=10:1; molar ratio). After that, the beads were washed with SELEX buffer 100 ul three times, and then ssDNA elution buffer (50 mM NaOH) 20 ul was added, and it was heated at 95° C. for 10 minutes, and ssDNA (single-stranded DNA) bound to the protein bound to beads was eluted. For the eluted ssDNA, symmetric PCR was performed under the following conditions, and thereby selected ssDNA was amplified:

(1) PCR mixture: Reverse primer(20 pM), biotinylated-forward primer(20 pM), dNTP(each 1 nM), pfu(+) buffer(CANCERROP Co., Ltd.), pfu(+) DNA polymerase (1U; CANCERROP Co., Ltd.), template-ssDNA(20 ng) prepared in Example 1;

(2) cycles: 95° C. for 5 min, start cycle (15 cycles)—95° C. for 30 sec, 53° C. for 30 sec, 72° C. for 10 sec, end cycle—72° C. for 5 min, 4° C. hold.

Forward primer:
5′-biotin-TAG GGA AGA GAA GGA CAT ATG AT 3 (23mer)
Reverse primer:
5′-TCA AGT GGT CAT GTA CTA GTC AA-3′ (23mer).

The dsDNA produced after performing PCR as above was used to obtain biotinylated-ssDNA (biotinylated-ssDNA for progressing SELEX 1 round: 86mer biotinylated-ssDNA prepared by commissioning to Bioneer Co., Ltd. described in Example 1; then, biotinylated-ssDNA for progressing SELEX 2 round and 3 round: biotinylated-ssDNA obtained by the process described below (method using streptavidin beads).

For the obtained PCR products, using streptavidin magnetic beads (NEB Co., Ltd.), biotinylated ssDNA was isolated.

More specifically, PCR product Mug obtained by the method and D.W, 200 mM NaOH, and 4× Wash/Binding Buffer (W/B buffer; 0.5 M NaCl, 20 mM Tris-HCl (pH 7.5), 1 mM EDTA (Ethylenediaminetetraacetic acid)) were mixed at a volume ratio of 1:1:1:1, and it was left at 37° C. for 10 minutes, to prepare PCR product solution (Final concentration: 50 mM NaOH, lx wash/binding buffer). On the other hand, streptavidin magnetic bead (NEB Co., Ltd.) solution 250 ul (pure bead amount: 1 mg) was taken to remove the supernatant, and it was washed with 1×W/B buffer 200 ul three times. The streptavidin magnetic bead in which the supernatant was removed was mixed well with the prepared PCR product (dsDNA state) solution 10 ug, and it was reacted at 37° C. for 1 hour to allow biotinylated-ssDNA to bind. After downing the streptavidin magnetic beads using a magnetic rack and then removing the supernatant, the beads were washed with 1×W/B buffer 200 ul three times. After putting D.W 200 ul to the biotinylated-ssDNA-bead obtained like this, it was left at 95° C. for 20 minutes to elute biotinylated-ssDNA. The ssDNA obtained like this was used for the next SELEX round.

### Example 4. Ka Value Measurement Through Indirect ELISA

The degree of binding (Ka value) between protein and the aptamer obtained for each protein by performing SELEX 3 round (See Example 3) by using the HFL or Random library prepared in Example 1 was measured by performing Indirect ELISA as follows.

Coating buffer (100 mM sodium carbonate/bicarbonate (pH 9.6), 3.03 g Na2CO3, 6.0 g NaHCO₃, 1000 ml distilled water) 100 ul and each protein 25 pM/well were loaded to a plate (Pierce 96-Well Polystyrene Plates, White Opaque), and it was incubated at a room temperature for 2 hours. Then, after removing the buffer in the plate by vigorously spraying it on the sink, 200 ul of wash buffer (PBS) was added to each well and it was washed 3 times repeatedly. Blocking buffer (5%(v/v) BSA in PBS) of 200 ul was added to each well and it was incubated at a room temperature for 3 hours. Then, after removing the blocking buffer in the plate by vigorously spraying it on the sink, 200 ul of wash buffer (PBS) was added to each well and it was washed three times.

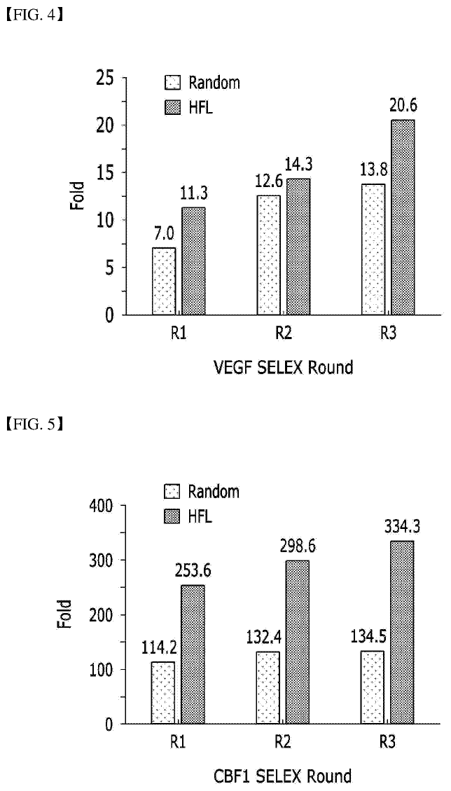

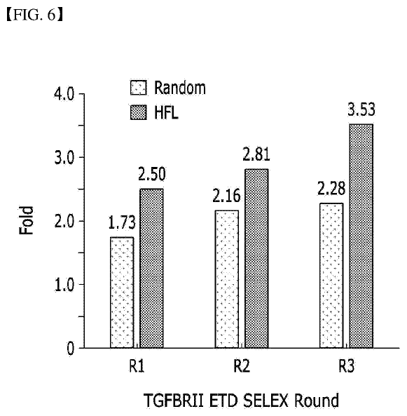

The biotinylated-ssDNA 0.2 pM selected per SELEX round in Example 3 was loaded with 100 ul of the SELEX buffer of each target protein (See Example 3) into each well of the prepared plate in which each protein was coated, and it was incubated at 37° C. for 1 hour (in the present example, 3 rounds were performed in total). After removing the solution in the plate by vigorously spraying it on the sink, 200 ul of wash buffer (PBS) was added to each well, and it was washed three times repeatedly. The streptavidin-HRP protein (abcam) was diluted in blocking buffer (5%(v/v) BSA in PBS) to 10000:1 (v:v), and 200 ul was loaded to each well, and then it was incubated at a room temperature under the darkroom condition for 2 hours. After removing the solution in the plate by vigorously spraying it on the sink, 200 ul of wash buffer (PBS) was added to each well, and it was washed three times. After that, 100 ul of ECL Substrate solution (SuperSignal ELISA Femto Substrate) was added to each well and the generated luminescence value was measured (measured by SPARK (TECAN)). For comparison, the luminescence value in case of no-treatment of the target protein was measured by the method the same as above and it was used as a background value. All tests were performed three times, and the average value was calculated and used for analysis of results. The result of dividing the obtained luminescence value by the background value was shown in FIG. 4 to FIG. 6.

As shown in FIG. 4 to FIG. 6, it could be confirmed that the aptamer obtained from the aptamer template having a stem-loop structure (HFL) had excellent binding ability to a target protein, even when performing a relatively less number of SELEX round (for example, 3 round), compared to the aptamer obtained from the conventional random library, and it could be confirmed that it showed high binding ability, for all the tested proteins having various pI values including protein having a low pI value with weak binding ability to the aptamer by electrostatic repulsive power (for example, TGFBRII ectodomain), compared to the aptamer obtained from the conventional random library.

1. A nucleic acid structure represented by the following general formula 2:
5′-[(N)ₚ][(X)₁][G(N)ₙ][(X)ₘ][(N)_{q}]-3′ (general formula 2)
wherein,
N is each independently selected from A, T or U, G, and C randomly, and
X is each independently selected from A, T or U, G, and C randomly, and the ratio of C or G is 55% or higher based on the total nucleotides of [(X)ₗ] or [(X)ₘ], and
[G(N)ₙ] is a site forming a loop structure, and n is the number of nucleotide N and an integer of 3 to 29, and
[(X)ₗ] and [(X)ₘ] are sites forming a stem structure by forming a base pair with antiparallel complementary sequences each other, and 1 and m are numbers of nucleotide X and each is an integer of 3 to 10, and
[(N)ₚ] and [(N)_{q}] are 5′ end region and 3′ end region, respectively, and p and q are numbers of nucleotide N, and each is independently an integer of 1 to 50, and p<q.

2. The nucleic acid structure according to claim 1,
wherein the [(X)ₗ] is 5′-GGAC-3′, and [(X)ₘ] is 5′-GTCC-3′ or 5′-GUCC-3′ which is anti-parallel complementary to the [(X)ₗ], and
the condition p<q is satisfied, and the p is selected from integers of 1 to 20, and the q is selected from integers of 15 to 50.

3. The nucleic acid structure according to claim 2, wherein the p is an integer of 1 to 10, and the q is an integer of 15 to 50.

4. The nucleic acid structure according to claim 1, wherein the nucleic acid structure is represented by the following general formula 3 or general formula 4:
(general formula 3; SEQ ID NO: 44)
NNNGGACGNN NNNNNGTCCN NNNNNNNNNN NNNNNNNNNN,
(general formula 4; SEQ ID NO: 45)
NNNGGACGNN NNNNNGUCCN NNNNNNNNNN NNNNNNNNNN,
wherein the N is each independently selected from A, T or U, G, and C.

5. An aptamer template comprising the nucleic acid structure of claim 1.

6. A composition for preparing an aptamer comprising the nucleic acid structure of claim 1.

7. A nucleic acid aptamer, prepared from the aptamer template of claim 5.

8. A method for preparing a target material-specific binding nucleic acid molecule, comprising
(1) contacting the nucleic acid structure of claim 1 with a target material; and
(2) measuring whether to bind between the nucleic acid structure and target material.

9. The method for preparing a target material-specific binding nucleic acid molecule according to claim 8, further comprising
(3) determining the nucleic acid structure as a nucleic acid molecule specifically binding to the target material, when binding between the nucleic acid structure and target material is confirmed.

10. The method for preparing a target material-specific binding nucleic acid molecule according to claim 8, wherein the nucleic acid structure is two or more kinds having different sequences each other.

11. The method for preparing a target material-specific binding nucleic acid molecule according to claim 10, wherein the target material-specific binding nucleic acid molecule is a target material-specific nucleic acid aptamer.

12. A method for preparing a nucleic acid aptamer specifically binding to a target material, comprising
(i) contacting a library comprising one or more kinds of the nucleic acid structures of claim 1 with beads in which a target material is immobilized;
(ii) washing the beads of the step (i); and
(iii) confirming the nucleic acid structure bound to the beads of the step (ii).

13. The method for preparing a nucleic acid aptamer according to claim 12, further comprising
(iv) determining the nucleic acid structure confirmed in the step (iii) as a nucleic acid aptamer specifically binding to the target material.

14. The method for preparing a nucleic acid aptamer according to claim 12, wherein the nucleic acid structure is two or more kinds having different sequences each other.

15. The method for preparing a nucleic acid aptamer according to claim 13, wherein the nucleic acid structure is two or more kinds having different sequences each other.
